(19) United States
(12) Patent Application Publication
(10) Pub. No.: US 2017/0127945 A1
(43) Pub. Date: May 11, 2017

(54) Respiratory Medicament and Therapy Data System and Method of Use

(71) Applicant: George Ashford Reed, Lake Oswego, OR (US)

(72) Inventor: George Ashford Reed, Lake Oswego, OR (US)

(21) Appl. No.: 14/938,805

(22) Filed: Nov. 11, 2015

(51) Int. Cl. A61B 5/00; A61M 11/00; G06F 19/00; A61B 5/08

(52) U.S. Cl. CPC A61B 5/0022; A61B 5/4848; A61B 5/0816; A61M 11/00; A61B 5/742; G06F 19/3418; A61B 2562/225

(57) ABSTRACT

A respiratory medicament and therapy data system capable of using a “breath actuated” monitoring device that monitors patient interactions with all respiratory therapy and drug delivery devices. It generates a signal based on the magnitude, direction and duration of the inhalation and exhalation pressures generated by a patient. This allows the tracking and wireless reporting of the various therapeutic and medicament delivery sessions with respect to such parameters as delivery date, time, duration, total delivered dose and the like. The respiratory medicament and therapy data system provides a fool-proof system for recording and immediately displaying the results of the respiratory therapy or drug delivery that have been algorithmically derived on a localized device and that can be sent to the medical provider's patient treatment database (health portals) for later, remote review by medical personnel.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF INVENTION

The present invention relates to a novel design for a respiratory medicament and therapy data system. It enables a new level of efficiency in charting patient progress, medicament delivery, patient follow-up, therapy session data and the like that is adapted to matingly connect with all standardized respiratory devices commonly utilized in all lung and respiratory disease treatment, testing, rehabilitation, medicament delivery and life support devices.

Currently patients with lung disorders and respiratory ailments undergo testing, medicament delivery, monitoring, phlegm dislodgment therapy, lung expansion therapy and other related medical procedures. Many of these are done in the hospital under medical supervision, while others are done unsupervised at home or are used in a hospital in conjunction with a ventilator. This may be a simple as a nebulizer aerosol medicament session or a positive exhalation pressure exercise (PEP) regime done at home. The problem the results of these go uncharted and possibly may be orally reported. The overall actual physical results of these are often charted however there is no ongoing record of each of these occurrences and what actually transpired on a lung inhalation basis for future trending analyses. This is a huge downfall of respiratory disorder treatment. Because they occur so frequently and often unsupervised, the overall effect or progress of these treatments is not always discernable, even to those medically trained in the industry. One glaring example of this, is the inability to actually know how often the patient is actually personally using their portable nebulizer or rescue inhaler. More on point, would be the inability of the respiratory disease treatment industry to tabulate the actual medicament delivered to a patient's lungs, or how often they really used their PEP device.

Henceforth, a respiratory medicament and therapy data system that can provide respiratory data to reflect the therapy sessions and treatments administered to a patient would fulfill a long felt need in the respiratory disease treatment industry. This new invention utilizes and combines known and new technologies in a unique and novel configuration to overcome the aforementioned problems and accomplish this.

### SUMMARY OF THE INVENTION

In accordance with the invention, the objects of the present invention, which will be described subsequently in greater detail, is to provide a respiratory medicament and therapy data system capable of using a “breath actuated” monitoring device that monitors patient interactions with all respiratory therapy and drug delivery devices.

It is a further object of this invention to provide a respiratory medicament and therapy data system that generates a signal based on values of the inhalation and exhalation pressures generated by a patient using a respiratory therapy, lung function test device or a drug delivery device.

It is another object of this invention to provide a respiratory medicament and therapy data system capable of tracking and wirelessly reporting the various sessions of therapy medicament delivery with respect to such parameters as delivery date, time, duration, total delivered dose and the like.

It is still another object of the present invention to provide a respiratory medicament and therapy data system capable of calculating and reporting the actual dose of medication delivered into a patient's lungs following an aerosol medicament delivery session.

It is still a further object of this invention to have respiratory medicament and therapy data system that may be powered by a battery, connect seamlessly and without any performance issues in a plethora of respiratory devices, and transmit its respiratory data wirelessly to a local computing device or smart phone with a wireless signal receiver.

It is a final object respiratory medicament and therapy data system to provide a fool-proof system for recording and immediately displaying the results of the respiratory therapy or drug delivery that have been algorithmically derived on a localized device and that can be sent to the medical provider's patient treatment database (health portals) for later, remote review by medical personnel.

The respiratory medicament and therapy data system has many of the advantages mentioned heretofore and many novel features that result in a new level of patient reporting which is not anticipated, rendered obvious, suggested, or even implied by any of the prior art, either alone or in any combination thereof.

The subject matter of the present invention is particularly pointed out and distinctly claimed in the concluding portion of this specification. However, both the organization and method of operation, together with further advantages and objects thereof, may best be understood by reference to the following description taken in connection with accompanying drawings wherein like reference characters refer to like elements. Other objects, features and aspects of the present invention are discussed in greater detail below.

### BRIEF DESCRIPTION OF THE DRAWINGS

FIG. 1 is a side perspective view of the smart adaptor;

FIG. 2 is a side perspective view of the smart adaptor with the protective housing removed;

FIG. 3 is an exploded view of the smart adaptor;

FIG. 4 is a cross sectional view of the smart adaptor without the protective housing;

FIG. 5 is a top view of the smart adaptor without the protective housing;

FIG. 6 is a left side view of the smart adaptor without the protective housing;

FIG. 7 is a bottom view of the smart adaptor without the protective housing;

FIG. 8 is a proximal end view of the smart adaptor without the protective housing;

FIG. 9 is a right side view of the smart adaptor without the protective housing;

FIG. 10 is a distal end view of the smart adaptor without the protective housing;

FIGS. 11-19 are views of the smart phone screen displays taken from various user input requests;

FIG. 20 is a flowchart of the operational steps of obtaining respiratory session data; and

FIG. 21 is a flowchart showing the operational steps of a pressure sensing event.

### DETAILED DESCRIPTION

The above description will enable any person skilled in the art to make and use this invention. It also sets forth the best modes for carrying out this invention. There are numerous variations and modifications thereof that will also remain readily apparent to others skilled in the art, now that the general principles of the present invention have been disclosed.

There has thus been outlined, rather broadly, the more important features of the invention in order that the detailed description thereof that follows may be better understood and in order that the present contribution to the art may be better appreciated. There are, of course, additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of descriptions and should not be regarded as limiting.

As used herein, the term “inhaled dose” refers to the amount of inhaled drug for a time interval of nebulization (generally one minute).

As used herein, the term “delivered drug dose” refers to the aggregate amount of aerosol medicament determined to have reached the patient's lungs in a single nebulizer treatment/session. This is determined knowing how many breaths the patient took, when they took the breaths, how long each inhalation lasted, the output of the aerosol generator per unit time, the concentration of the drug and any attenuation/efficiency factors taken into consideration.

As used herein, the term “nebulizer system” refers to a respiratory medicament aerosol generator.

As used herein, the terms “microprocessor” or “logic chip” means a computer processor on a microchip that contains all, or most of, the central processing unit (CPU) functions and is the “engine” that goes into motion when the pressure sensor sees a change in the pressure exerted upon it. It incorporates a real time clock and either or both of volatile/non volatile memory and performs arithmetic and logic operations based on input signals or data from remote devices such as the pressure transmitter, or manually operated electrical switches. It outputs operational signals that integrates with other electrical circuits. It may also output algorithmically derived data to an external computing device. (This may be a local computer, smart phone, or a health provider's network via a remote server.) These operations are the result of a set of instructions that are part of the microprocessor design as is well known in the industry. In simple terms, the microprocessor is a multipurpose, programmable device that accepts digital data as input, processes it according to instructions stored in its memory, and provides results as output.

As used herein the term “pressure sensor” means an electromechanical device that converts an external pressure (or a change in a the external pressure) of a gas or fluid exerted on the sensor, into an electrical signal. The electrical signal generated is a function of the pressure exerted on the sensor. Pressure sensors are also commonly called pressure transducers, pressure transmitters, pressure senders, pressure indicators, piezometers and manometers, among other names. Common types of pressure sensors are piezoresistive strain gauges, capacitive, electromagnetic, piezoelectric, optical and potentiometric.

As used herein, the term “personal mobile device” refers to a device that is both portable and capable of collecting, storing, transmitting or processing electronic data or images. Examples include laptops or tablet PCs, personal digital assistants (PDAs), and “mobile smart” phones. This definition also includes storage media, such as USB hard drives or memory sticks, SD or CompactFlash cards, and any peripherals connected to the device.

As used herein, the term “mobile smartphone” means any web-enabled mobile phone. While the term “smartphone” is well known in the art, smartphones typically include a touch sensitive screen, a messaging client, global positioning systems (GPS) technology or any other geo-position mechanisms to determine the physical coordinates of the smartphone, and a browser application. The browser application employs any web-based language such as JavaScript Object Notation (JSON), JavaScript, HyperText Markup Language (HTML), or any other web-based programming language capable of sending and displaying messages, search queries, and search query results.

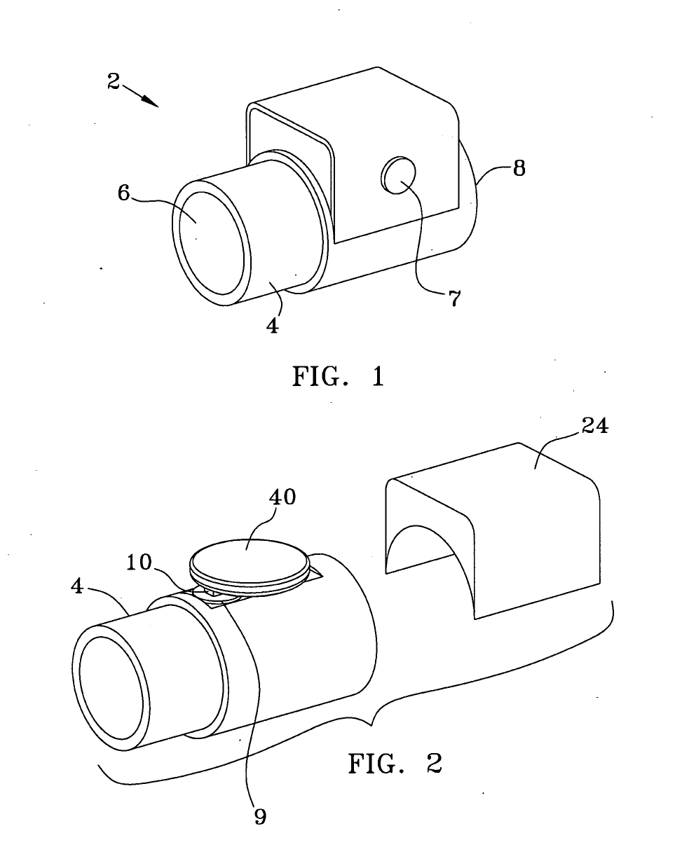

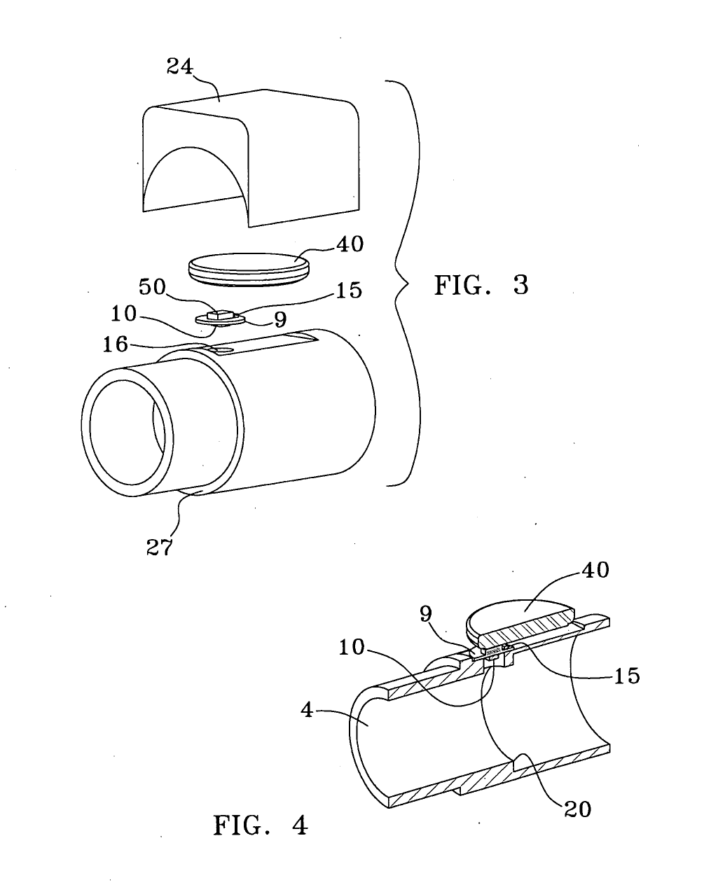

Looking at FIGS. 1 and 2, the smart adaptor 2 of the respiratory medicament and therapy data system can best be seen. It has a generally hollow tubular, circular adaptor body 4 having an unobstructed, open proximal end 6 and an unobstructed, open distal end 8, so as to allow the unhampered passage of respiratory gases and medicaments therebetween. As illustrated in FIGS. 3 and 4, in the approximate center of the body 4 is an orifice 16 into which part of the “smart” assembly is affixed. The “smart assembly” is a breath monitoring and wireless reporting device. This “smart” assembly is a miniaturized pressure sensor 10 mounted on a printed circuit board 9 (which is generally laminar, non-conductive substrate) that mechanically supports and electrically, operationally connects all of the electronic components of the smart assembly. This includes the wireless transmitter's integrated circuitry, a battery 40, a micro switch 15, (FIG. 9) the pressure sensor 10 and the microprocessor 50 necessary for functionality as is well known in the industry. (Generally, this battery will be of a “coin” style configuration.) There is a detachable battery 7 that functions to activate the micro switch 15. There is a related health care management software application (Welltrac) that is a set of computer instructions intended for downloading onto a personal mobile device (generally a cellular smart phone or computer tablet) however it may be operated on any computing device. This software enables the microprocessor and memory in the cell phone to interact with the incoming wireless data sent from the smart adaptor 2 as well as any of its own data such as time and date, and run any software program algorithms, tabulate, store, format and display the aggregated data in a medically informative format. Basically, once installed to a mobile personal device it will allow the transmission, reception, interpretation and display of data from said breath monitoring and wireless reporting device to the personal mobile device.

It may act as a counter, considering only separate occurrences of inhalation/exhalation, or it may consider the duration and magnitude of the inhalation/exhalation pressures for additional respiratory determinations.

The only component that will be in physical contact with the gases/fluid flowing through the body 4 will be the pressure sensor 10. The pressure sensor 10 will be sealed to or about the perimeter of the orifice 16 by its tight mechanical interference fit with an external peripheral seal of silicone or other resilient material so as to remain in operational contact with the respiratory gases. This seal may reside on the pressure sensor 10, on the PCB plate 9, in or adjacent the orifice 16 on the body 4. The other components of the smart assembly 2 will be encased in a protective housing 24. In this way the body 4 may be cleaned from time to time with water and dish soap or isopropyl alcohol without damage to the electronics.

The pressure sensor 10 of the preferred embodiment is a gauge pressure sensor that measures the pressure relative to atmospheric pressure. Thus when it indicates zero, then the pressure it is measuring is the same as the ambient pressure. Other types of pressure sensors may be utilized such as sealed pressure sensors or differential pressure sensors as is well known in the industry. These may require modifications to the body 4 as would be well known by one skilled in the art.

The pressure sensor selection has taken into consideration that it must detect an inhalation pressure (negative pull) of <(−) 2 cm H2O and when used in a positive pressure application, the pressure sensor will be exposed to positive peak pressures in excess of (+) 90 cm H2O. The pressure sensor will be exposed to temperatures and humidity consistent with human respiration; 98.6° F./37° C. at 98% RH for 30 minutes 3-4 times per day. The senor tip may come in contact with respiratory therapy medications and human saliva. The pH of the respiratory drugs range from 3.5 to 7.4. The pH of saliva is 6.5-7.5. The operating temperature ranges from 20-50 degrees C. and the cleaning temperature ranges from 20 to 110 degrees C.

It is the one of the goals to have the body 4 designed for fool-proof connection. Since the operation of the pressure sensor 10 is not affected by the direction of the gaseous flow through the tubular body 4, the body cannot be installed in a wrong direction. There is thus no need for directional arrows on the body 4. The pressure sensor 10 resides in orifice 16 but the wall thickness of the body 4 in that region is chosen to be thicker than the depth of the pressure sensor 10 such that the pressure sensor 10 does not extend into the flow cavity of the body 4. In this way, while the pressure sensor 10 may be covered by an extreme insertion of a respiratory device, it cannot be damaged by such action.

However, there are two remaining areas where installation could be a problem. The first occurs when during the installation, the mated respiratory device is inserted too far into the body 4 so as to cover the internal opening 16 for pressure sensor 10 (rendering it inoperable) or jam up against the housing for the smart assembly and dislodging or operationally separating any of its components (rendering it inoperable and possibly unrepairable). Obviously, this would not be a problem if the length of the body 4 were extreme, e.g. in the range of 6 inches or more, but this is not practical, as when the length of the adapter increases so does the internal surface area which allows for more condensation of aerosol medicament, resulting in a lesser amount of medicament reaching the patient's lungs. Also, as the length increases, the frictional losses for gas flowing through the body 4 proportionally increase. This can disrupt any other readings being taken by connected respiratory equipment or reduce the strength of the pressure wave reaching a patient's lung from a PEP therapy session. Neither of these are desirable effects. For this reason the body's overall length is minimized, miniaturized components are used and the only end sized for insertion of a respiratory apparatus, is the distal end 8 which sees an interior abutment stop 20 before the orifice 16. (FIG. 10) This safeguards the smart assembly from physical damage by the unintended abutment of a mated respiratory device into the body 4 so as to cover the pressure sensor 10.

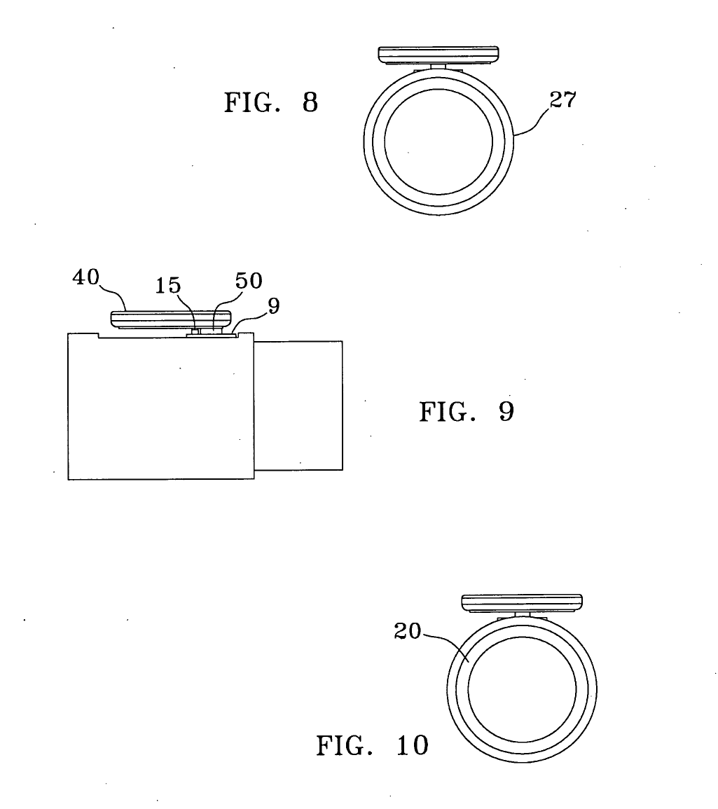

In the same way, there is an exterior abutment stop 27 adjacent the proximal end 6 to protect the over insertion of the smart adaptor 2 into a respiratory device. (FIG. 8) In the preferred embodiment the body 4 has an overall length from distal end 8 to proximal end 6 of approximately 46 mm.

The proximal end's male fitting configuration is designed to accept connection of a respiratory device on or inside of its circular body. The distal end's female socket configuration is designed for connection of a respiratory device by its insertion into the inside of its body. Looking at FIGS. 4, 8 and 10 it can be seen that the external diameter of the male fitting approximates the internal diameter of the female socket. The male fitting supports an 18 mm diameter internal connection and an external diameter that supports a 22 mm diameter external connection (it can fit internally into a 22 mm diameter connection.) The female fitting has an internal diameter that supports a 22 mm diameter fitting. In this way the body 4 may be connected in a plethora of different ways to the most common sizes of respiratory fittings.

In its simplest form, the respiratory medicament and therapy data system consists of an adaptor 2 fitted with an electronic pressure sensor 10 and a wireless transmitter powered with a coin battery 40 and operatively connected on a substrate printed circuit board 9, that act to count the breaths taken by a patient and wirelessly report this number to a health portal (such as Microsoft HealthVault or Apple Health Kit) or to a personal mobile device that has downloaded and runs the companion mobile software application (Welltrac) thereon that tabulates, records in memory, and displays this data as a health management tool. Optionally, the actual pressures sensed and their duration will be wirelessly sent for a more intricate level of medical result reporting.

In the In the preferred embodiment the wireless transmission is done via Bluetooth wireless technology protocol (using short-wavelength UHF radio waves in the ISM band from 2.4 to 2.485 GHz) although other protocols may be substituted such as ultra wideband or induction wireless, to name a couple. The preferred embodiment uses a Bluetooth Dialog Semiconductor SmartBond (DA14580) and a SM1120-1100H-A-G or SM9520-040M-G-D SMi pressure sensor. The power switch is a Magnetic KSK-14OA Series Reed switch. It has a normally closed (NC) configuration that will control the power to the integrated circuit. The smart adaptor 2 will come packaged with an appropriately matched magnet affixed to its exterior that will hold the NC micro reed switch in the open position. When the magnet is removed the switch will revert to the NC position and the circuit will be activated. Replacing the magnet will turn off the smart adaptor 2. The battery will be a Lithium coin style battery with a nominal voltage of 3.0 volts, generally designated in the industry as a CR1025 battery.

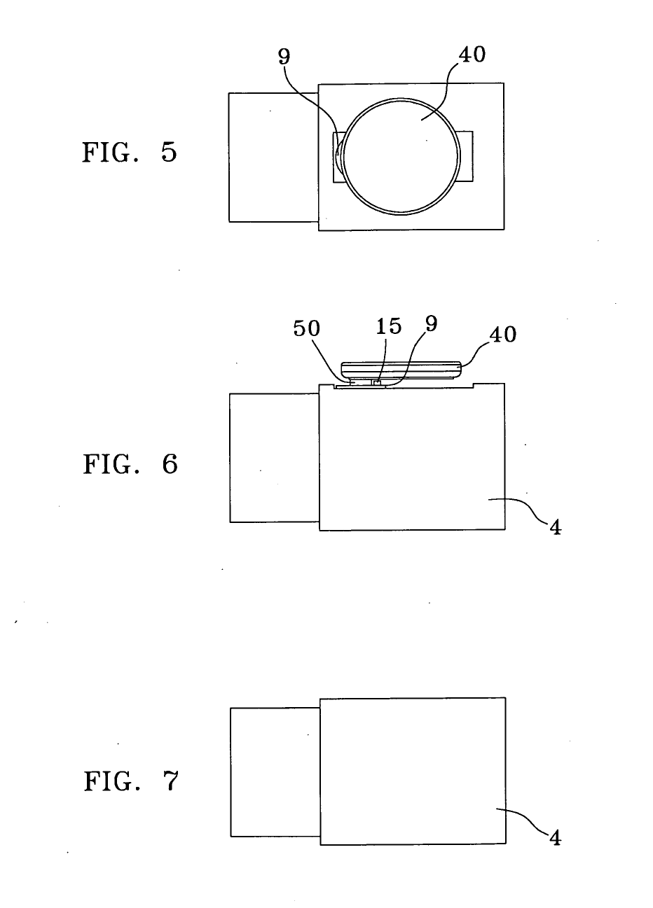

Dimensionally, as stated earlier the electrical components are miniaturized. In the preferred embodiment the pressure sensor will be in the 700 micron×220 micron×75 micron range, the coin battery will be in the 20 mm diameter with a 3.2 mm thickness range, the wireless transceiver will have dimensions in the 2.5 mm×2.5 mm×0.5 mm range and the power switch will have a length of 4.1 mm and a diameter in the 1.25 mm range. All components will fit onto a substrate with a maximum dimension of 15 mm×15 mm. With all components operationally assembled on substrate, the thickness will be less than 5 mm. The silicon plug to seal the pressure sensor will have a diameter of approximately 2.5 mm. FIGS. 5-7 and 9 illustrate the general spatial arrangement and relative sizing of the smart adaptor 2.

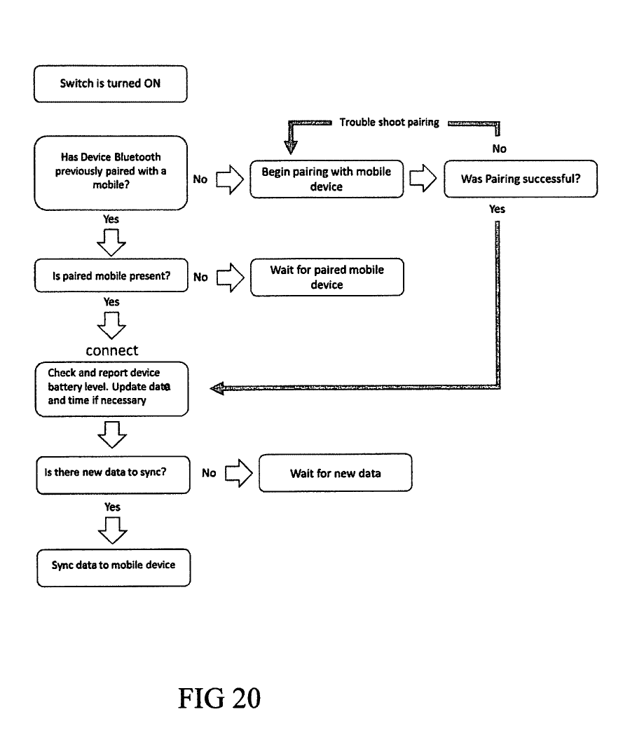

In operation, the system works in the following way. (FIG. 20) The personal mobile device accesses the internet and downloads the respiratory health kit software application (Welltrac) into its memory for integration with its operating system such that all of the application's instructions and algorithms are functionally operational. The smart adaptor 2 is fit between a respiratory treatment or therapy device and its mouthpiece. (Although placement elsewhere in the respiratory circuit may also allow for full functionality.) The exterior, detachable magnet 7 is removed from the smart adaptor 2 such that the micro switch 15 powers up the smart adaptor 2. The smart adaptor 2 utilizing Bluetooth handshake technology, is paired with any other compatible local Bluetooth device it had been previously paired with. At this stage there is none. The personal mobile device is set to the bluetooth ‘pairing mode’ and finds the smart adaptor 2 and connects the two for wireless communication via the connection protocol for that personal mobile device's operating system. The health care management software application (Welltrac) is accessed on the personal mobile device and its operational, interactive screens and visual prompts are shown on the display. The smart adaptor's battery level is optionally displayed on the personal mobile device. A window for the input of data (initial or updated) about the date, time, patient (name, age, sex, weight, height, race, and the like) respiratory type of sessions, scheduled sessions, type of respiratory device used, and optionally, the doctor's data, medicament and medicament specifics (i.e. concentration) The smart adaptor 2 sends respiratory session data to the personal mobile device as it is available. This is accomplished by industry standard wireless data transfer protocol that includes receipt verification prior to data erasure from the smart adaptor as is well known in this field of art.

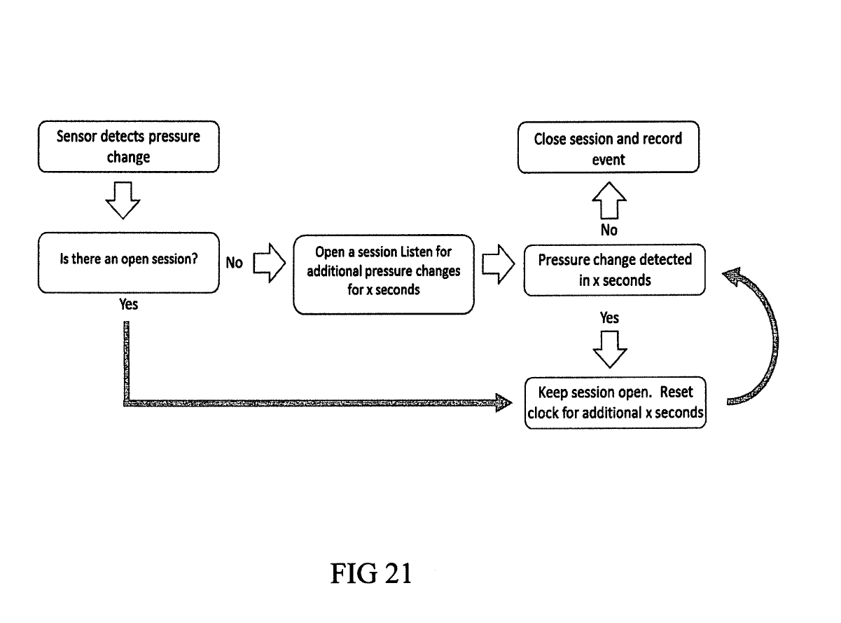

Looking at FIG. 21 the operational logic for the transfer of data from the smart adaptor 2 to the personal mobile device is set forth in flowchart format. When powered, the smart adaptor 2 sits dormant until the sensor 10 detects a pressure change. If there is an ongoing session its adds the data to that session (stored in the smart adaptor's memory) and resets its timer. If the pressure sensor 10 does not detect any further pressure changes before the timer expires, the session is closed and the complete data set for that session is sent to the personal mobile device for interpretation, storage and display. This transfer of data follows any of the conventional data transfer and verification “handshake” protocols commonly utilized and well known in the industry. If there is another pressure change detected before the timer expires it is added to the session and the timer is reset. Once the session is ended and the data is sent to the personal mobile device, after the data transfer and verification protocol is completed, that data is marked as sent, and earmarked for eventual deletion from the smart adaptor. It is to be noted that the smart adaptor 2, having its own memory may be used without connection to a personal mobile device. (It is capable of storing numerous sessions and retaining them for eventual transfer upon pairing with a suitable personal mobile device.) Upon the establishment of the Bluetooth connection, if there is any session data on the smart adaptor 2 not marked as sent, it will be relayed to the personal mobile device. (See FIG. 21) The compiled data is saved to memory, displayed and optionally sent to another computing device via email or synched to a health portal where it can be tagged to the patients medical record. In this manner patients and caregivers can see and track their respiratory progress. (The health portal is accessed through the personal mobile device. On the initial setup of the Weltrac application on the personal mobile device, the user will define an account or profile for that patient, given a username and password. on a dedicated Welltrac server portal. Upon establishment of this Welltrac account, the respiratory data may be transferred for storage to this server portal. This server portal has the communication capability to link with the various healthcare information portals so as to enable the transfer of that patient's data into that patient's discrete medical file.)

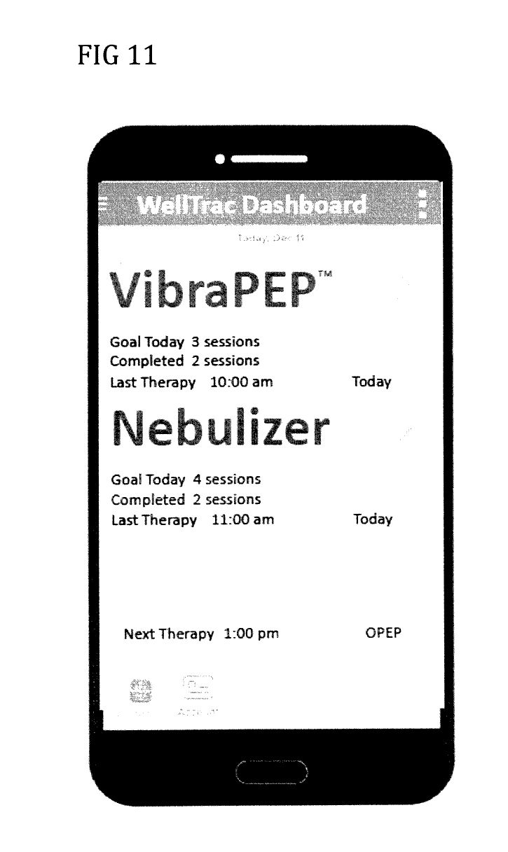

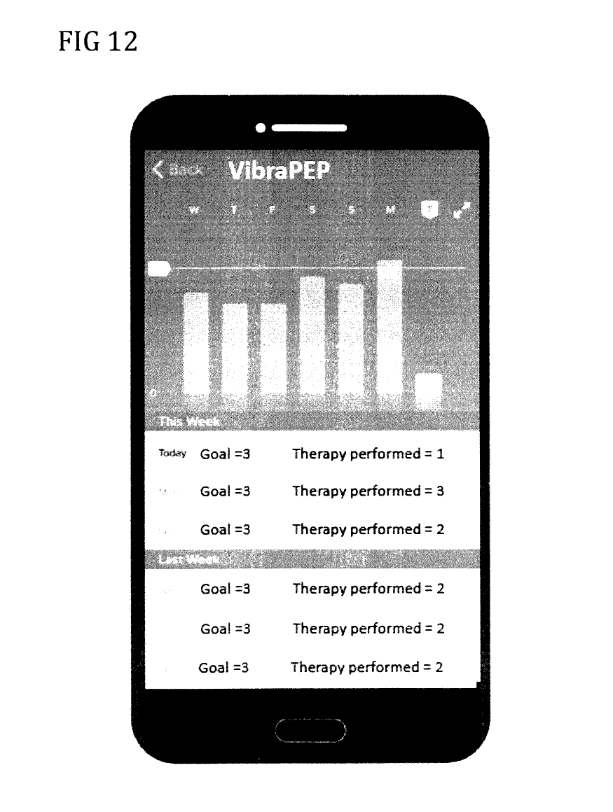

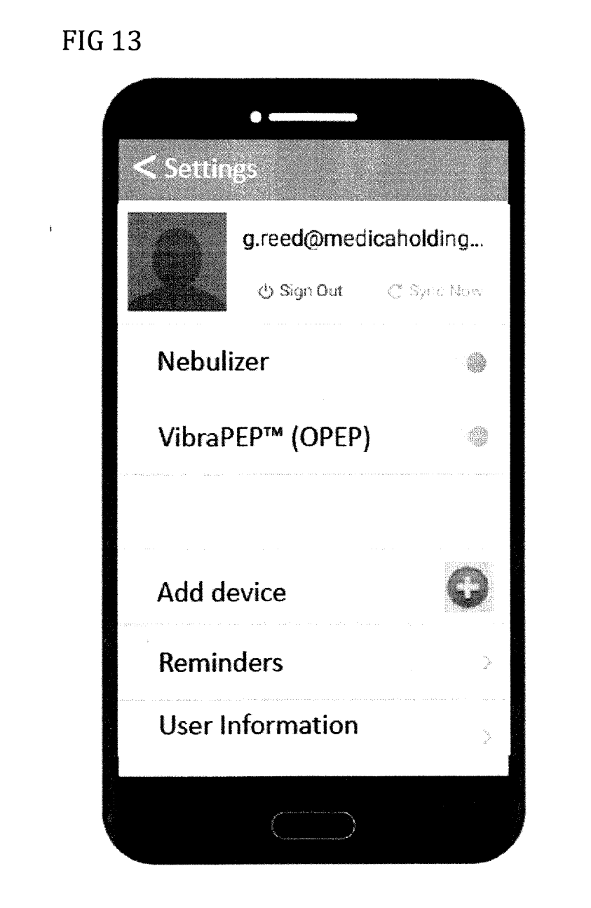

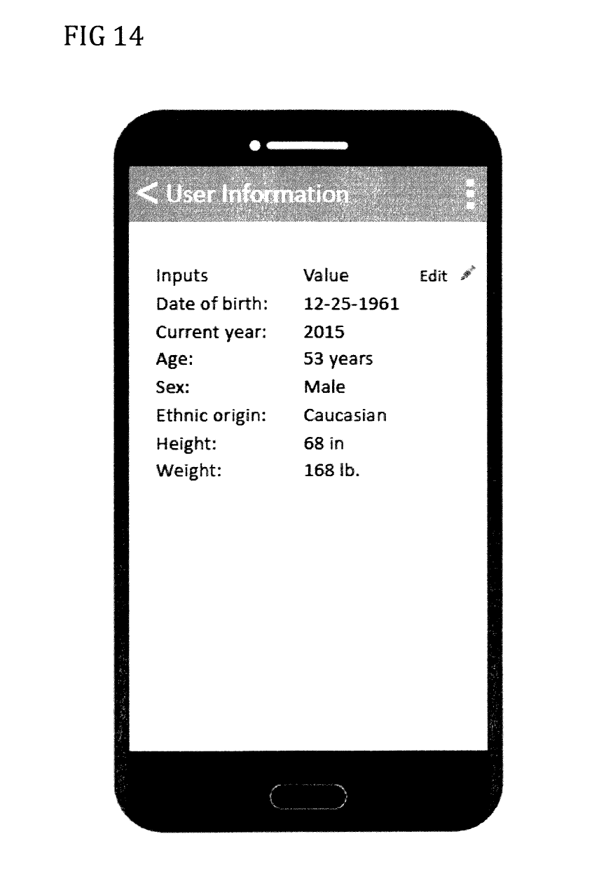

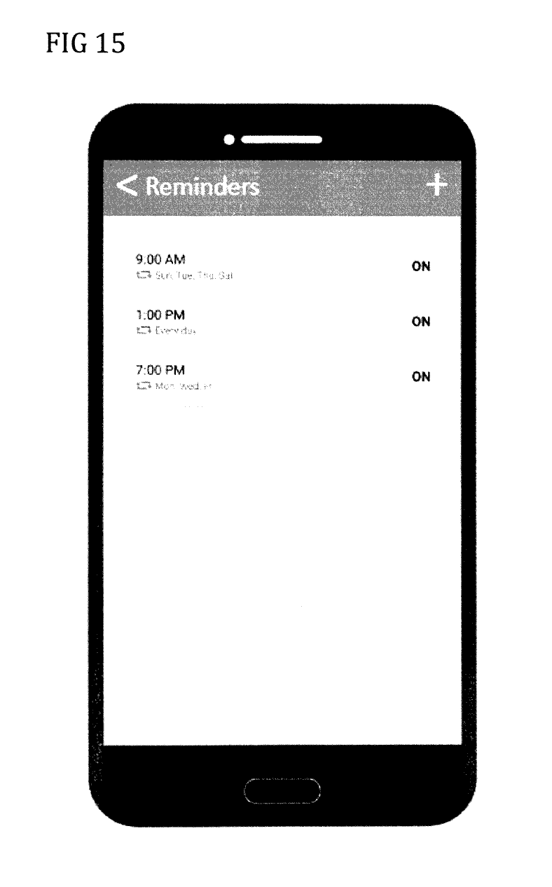

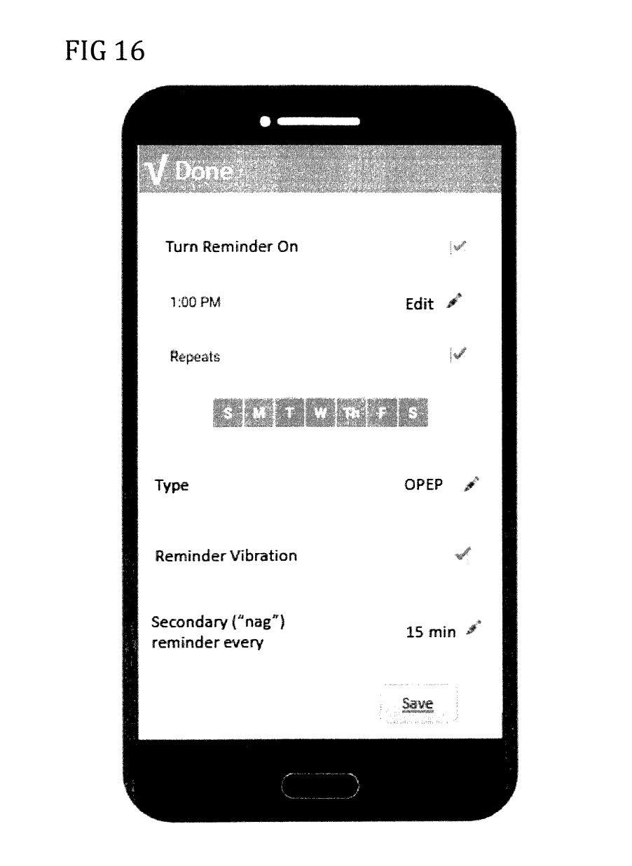

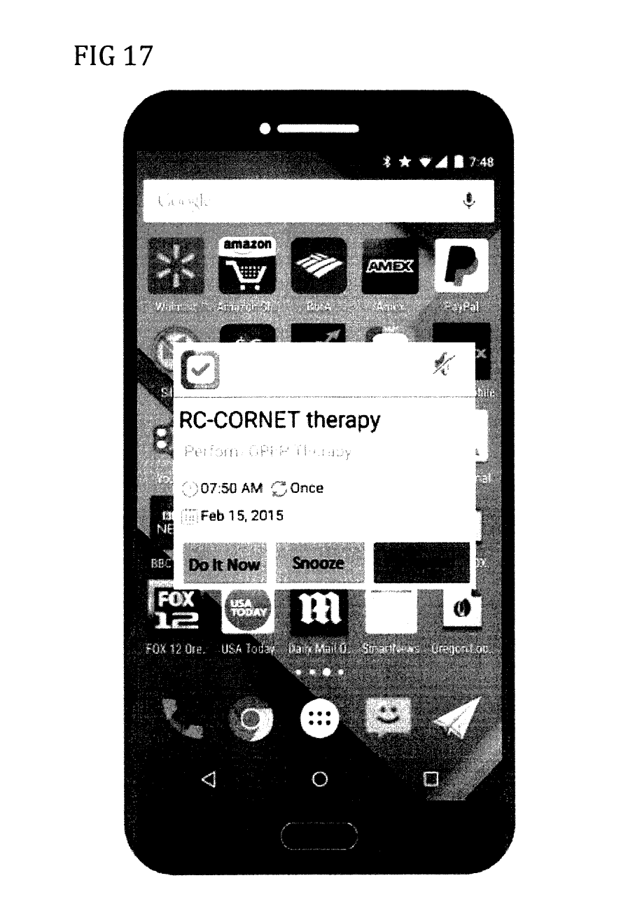

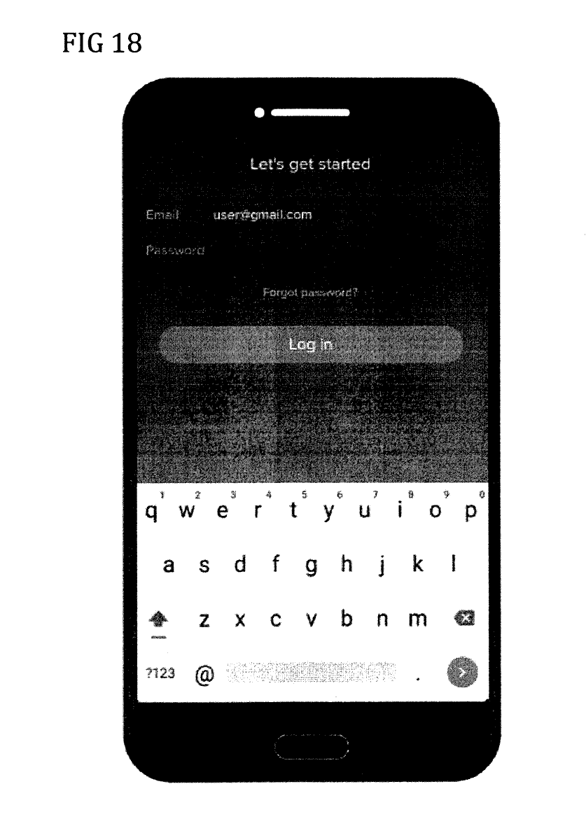

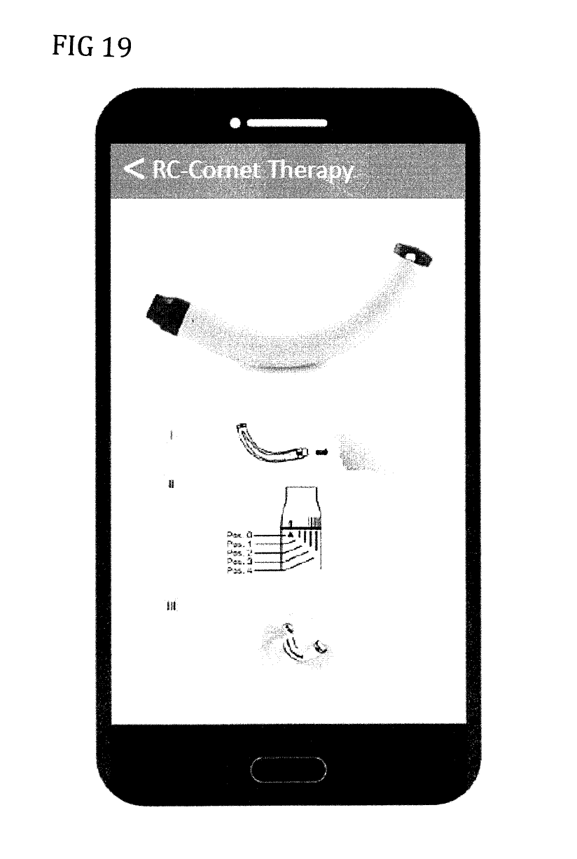

The respiratory health kit software application (Welltrac) when accessed on the personal mobile device, and logged in (FIG. 18) allows the user, the interactive display of user information (FIG. 14), reminder input (FIG. 16), active reminders (FIG. 15), session schedule status (FIG. 11), respiratory therapy equipment selection (FIG. 13) session history (FIG. 12), and equipment instructions (FIG. 20), smart adaptor battery status, last date and time of synchronization, progress status based on input goals. The Welltrac application also initiates session reminders (FIG. 17) in an alarm function even when it is not not active on the display screen. It is to be noted that the user assignment of the type of respiratory device that the smart adaptor 2 is connected (FIG. 13) (e.g. nebulizer, PEP device, OPEP etc.) is critical to the personal mobile device's properly analyzing and reporting the respiratory therapy session. The actual identification of the type of respiratory device may also be transmitted to the personal mobile device by a wireless transmitter on the respiratory device, when available.

When the Welltrac software application is accessed it initiates the personal mobile device to attempt to detect other nearby devices operating the same type of wireless technology (such as the smart adaptor 2) and electronically “pair it” with the personal mobile device. (Multiple smart adaptors may be paired on the same personal mobile device.)

It is noteworthy that smart adaptor may be fabricated in various levels to work with the health kit software application (Welltrac). In the most sophisticated version when the patient inhales or exhales, the smart adaptor 2 will sense the change in atmospheric pressure whether it be a decrease or increase in pressure over the ambient atmospheric pressure and it will send a wireless signal to the paired personal mobile device that is proportional to the magnitude and direction of the pressure change throughout the duration of the pressure change. In this level the personal mobile device will open a reporting session that will record the date, the time, the magnitude and direction of the pressure changes, the number of pressure changes, and the length of the pressure changes. Using algorithms from the software application, and optionally input data (such as medicament, medicament concentration etc.) the personal mobile device will interpret the results of the session into a displayable format for the selected type of respiratory session previously chosen. This may include data as to the strength of the breath pulses, the duration, the amount of medicament received, timing between breaths and the like. This above and beyond what interpretive data the basic level of smart adaptor generates. The basic level merely counts the breath pulses, compiling them into sessions and sends them to the personal mobile device where they will show if the scheduled session was done, when for how long and the number of breaths taken.

In simplified terms the method of reporting data from a the breath monitoring and wireless reporting device comprises the steps of:

installing a breath monitoring and wireless reporting device software application onto a personal mobile device;

connecting a smart adaptor having a breath monitoring and wireless reporting device to a respiratory medicament delivery and/or respiratory therapy device;

powering on said breath monitoring and wireless reporting device of said smart adaptor;

allowing a pressure sensor to generate a data set of the number of breaths taken by a patient during a respiratory medicament delivery session or a respiratory therapy session;

storing said data set in a memory of said breath monitoring and wireless reporting device;

pairing said personal mobile device to said smart adaptor and establishing a wireless communication between said smart adaptor and said personal mobile device;

transferring said data set to an operating system of said personal mobile device utilizing a data transfer protocol with a data receipt acknowledgment;

interpreting said data set and generating a set of results on said personal mobile device; and

providing a visual display of said set of results on a screen of said personal mobile device.

Optionally, the step of allowing a pressure sensor to generate a data set of the number of breaths taken by a patient during a respiratory medicament delivery session or a respiratory therapy session, may also include associating the breaths taken with a time and date, a magnitude, a duration and a direction, and incorporating into said data set.

Those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Having thus described the invention, what is claimed as new and desired to be secured by Letters Patent is as follows:

1. A respiratory medicament and therapy data system comprising:
a smart adaptor having an adaptor body with a breath monitoring and wireless reporting device; and
a breath monitoring and wireless reporting device software application for installation to a mobile personal device to allow transmission, reception, interpretation and display of respiratory data sets from said breath monitoring and wireless reporting device to a personal mobile device;
wherein said adaptor body has an open distal end and an open proximal end to allow the unhampered passage of respiratory gases and medicaments there between; and
wherein an orifice is formed in said adaptor body, there between said proximal end and said distal end; and
wherein said breath monitoring and wireless reporting device has a pressure sensor operationally connected thereon so as to generate respiratory data sets; and
wherein said breath monitoring and wireless reporting device is affixed to said adaptor body and said pressure sensor is positioned adjacent said orifice so as to reside in operational contact with said respiratory gases.

2. The respiratory medicament and therapy data system of claim 1 wherein said respiratory data set is selected from the set of respiratory data consisting of date, time, magnitude and direction of pressure changes, number of pressure changes, and duration of pressure changes.

3. The respiratory medicament and therapy data system of claim 1 wherein said adaptor body has an exterior abutment stop adjacent said proximal end, and an interior abutment stop between the distal end and said orifice.

4. The respiratory medicament and therapy data system of claim 1 wherein said breath monitoring and wireless reporting device has further components of:
a microprocessor;
a wireless transmitter;
a battery;
a micro switch;
a printed circuit board;
wherein said printed circuit board mechanically supports and operationally connects all components of said breath monitoring and wireless reporting device.

5. The respiratory medicament and therapy data system of claim 1 wherein said distal end accommodates a 22 mm diameter internal connection, and said proximal end accommodates an 18 mm diameter internal connection and also accommodates a 22 mm diameter external connection.

6. The respiratory medicament and therapy data system of claim 4 wherein said micro switch is magnetically actuated.

7. The respiratory medicament and therapy data system of claim 4 further comprising a cover affixed to said adaptor body, wherein said cover houses said breath monitoring and wireless reporting device therein.

8. The respiratory medicament and therapy data system of claim 1 wherein said pressure sensor is an electronic, gauge pressure sensor.

9. A method of reporting data from a breath monitoring and wireless reporting device comprising the steps of:
installing a breath monitoring and wireless reporting device software application onto a personal mobile device;
connecting a smart adaptor having a breath monitoring and wireless reporting device to a respiratory medicament delivery and/or respiratory therapy device;
powering on said breath monitoring and wireless reporting device of said smart adaptor;
allowing a pressure sensor to generate a data set of the number of breaths taken by a patient during a respiratory medicament delivery session or a respiratory therapy session;
storing said data set in a memory of said breath monitoring and wireless reporting device;
pairing said personal mobile device to said smart adaptor and establishing a wireless communication between said smart adaptor and said personal mobile device;
transferring said data set to an operating system of said personal mobile device utilizing a data transfer protocol with a data receipt acknowledgment;
interpreting said data set and generating a set of results on said personal mobile device; and
providing a visual display of said set of results on a screen of said personal mobile device.

10. The method of reporting data from a breath monitoring and wireless reporting device of claim 9 comprising the further step of:
associating said breaths taken by a patient during a respiratory medicament delivery session or a respiratory therapy session with a time and date and incorporating into said data set.

11. The method of reporting data from a breath monitoring and wireless reporting device of claim 10 comprising the further step of:
associating said breaths taken by a patient during a respiratory medicament delivery session or a respiratory therapy session with a magnitude and direction and incorporating into said data set.

12. The method of reporting data from a breath monitoring and wireless reporting device of claim 11 comprising the further step of:
associating said breaths taken by a patient during a respiratory medicament delivery session or a respiratory therapy session with a duration and incorporating into said data set.
